(19) United States
(12) Patent Application Publication
(10) Pub. No.: US 2023/0097931 A1
(43) Pub. Date: Mar. 30, 2023

(54) METHOD FOR TREATING CHRONIC GRAFT VERSUS HOST DISEASE

(71) Applicant: Mesoblast International Sárl, Meyrin (CH)

(72) Inventor: Silviu ITESCU, Melbourne (AU)

(21) Appl. No.: 17/904,581

(22) PCT Filed: Feb. 18, 2021
(86) PCT No.: PCT/EP2021/054066

§ 371 Date: Aug. 18, 2022

(30) Foreign Application Priority Data
Feb. 19, 2020 (AU) 2020900472

(51) Int. Cl. A61K 35/28; A61K 35/16; A61K 47/42; A61K 47/20; A61P 37/06

(52) U.S. Cl. CPC A61K 35/28; A61K 35/16; A61K 47/20; A61K 47/42; A61P 37/06

(57) ABSTRACT

The present disclosure relates to methods for treating or preventing chronic graft versus host disease in a subject in need thereof, the method comprising administering to the subject a composition comprising culture expanded mesenchymal lineage precursor or stem cells (MLPSCs).

### FIELD OF THE DISCLOSURE

The present disclosure relates to methods for treating or preventing chronic graft versus host disease in a subject in need thereof.

### BACKGROUND

Acute and chronic Graft versus Host Disease (GvHD) are immunological disorders that are major factors which limit the success and availability of allogeneic stem cell transplantation. Both acute and chronic GvHD are associated with a systemic inflammatory reaction which causes chronic illness and may lead to death of the host mammal. However, over the past several years, chronic GvHD (cGvHD) has emerged as the most troublesome complication of allogeneic bone marrow or stem cell transplantation.

cGvHD is distinct from acute GvHD (aGvHD). It is well known that aGvHD and cGvHD involve distinct pathological processes aGvHD is thought to be mainly Th1/Th17 driven process and to have strong inflammatory components whereas cGvHD displays more autoimmune and fibrotic features.

B cells have increasingly been recognized in recent years as major factors in cGvHD. For example, after stem cell transplantation, patients with cGvHD have an obviously impaired reconstitution of CD5+ B cells.

Trials of mesenchymal stem cells (MSCs) in patients with refractory cGvHD have observed variable outcomes. Accordingly, there remains an unmet therapeutic need in patients with cGvHD and/or its associated symptoms with new treatment options being required.

### SUMMARY OF THE DISCLOSURE

The present inventors have surprisingly identified that early disease response (day 28) can be achieved in subjects with chronic Graft versus Host Disease (cGvHD) by administration of culture expanded mesenchymal lineage precursor or stem cell that have been cryopreserved and thawed. These mesenchymal lineage precursor or stem cells constitute an “off the shelf” product and there is no requirement for HLA matching of the mesenchymal lineage precursor or stem cell donors and cGvHD patients.

Accordingly, in a first example, the present disclosure relates to a method of treating or preventing chronic Graft versus Host Disease (cGvHD) in a human subject in need thereof, the method comprising administering to the subject a composition comprising culture expanded mesenchymal lineage precursor or stem cells (MLPSCs).

In an example, the culture expanded MLPSCs have been cryopreserved and thawed. Accordingly, in another example, the present disclosure relates to a method of treating or preventing chronic Graft versus Host Disease (cGvHD) in a human subject in need thereof, the method comprising administering to the subject a composition comprising culture expanded mesenchymal lineage precursor or stem cells (MLPSCs), wherein the culture expanded MLPSCs have been cryopreserved and thawed.

In an example, the MLPSCs are culture expanded from an intermediate cryopreserved MLPSCs population. In another example, the MLPSCs are culture expanded for at least about 5 passages.

In an example, the MLPSCs express at least 13 pg TNFR1 per million MLPSCs. In an example, the MLPSCs express about 13 pg to about 44 pg TNFR1 per million MLPSCs.

In another example, culture expansion comprises at least 20 population doublings. In another example, culture expansion comprises at least 30 population doublings.

In an example, the subject is refractory to steroid immunosuppressant and/or a biologic therapy. In another example, the subject has at least a partial response after 28 days of treatment. In another example, the subject has at least a partial response at least 28 to 90 days after treatment. In an example, a partial response is characterized by one or more or all of:
Reduction in Skin % BSA score of at least one point;
Reduction in mouth score of at least one point;
Reduction in eye score of at least one point;
Reduction in skin features score of at least one point;
Reduction in gastrointestinal tract score of at least one point;
Reduction in liver score of at least one point;
Reduction in lung symptom score of at least one point;
Reduction in lung FEV1 score of at least one point;
Reduction in joints and fascia score of at least one point;
Reduction in genital tract score of at least one point.

In another example, a partial response is characterized by one or more or all of:
Reduction in Skin % BSA score of at least one point;
Reduction in mouth score of at least one point;
Reduction in eye score of at least one point.

In another example, the MLPSCs are administered intravenously. In an example, the MLPSCs are mesenchymal stem cells (MSCs). In another example, the MLPSCs are allogeneic.

In another example, the methods of the disclosure comprise administering between 10 x 10⁶ and 2 x 10⁸ cells per dose. In another example, the methods of the disclosure comprise administering between 20 x 10⁶ and 1 x 10⁷ cells per dose. In another example, the subject receives at least two doses. In another example, the subject receives at least 2, 3, 4, 5, 6, 7, 8, 9 or 10 doses. In an example, the first two doses are administered weekly for two weeks. In another example, the first two doses are administered weekly every two weeks. In another example, third and subsequent doses are administered monthly.

In an example, the composition further comprises Plasma-Lyte A, dimethyl sulfoxide (DMSO), human serum albumin (HSA). In another example, the composition further comprises Plasma-Lyte A (70%), DMSO (10%), HSA (25%) solution, the HSA solution comprising 5% HSA and 15% buffer.

In an example, the composition comprises greater than 6.68x10⁶ viable cells/mL.

### DETAILED DESCRIPTION

Throughout this specification, unless specifically stated otherwise or the context requires otherwise, reference to a single step, composition of matter, group of steps or group of compositions of matter shall be taken to encompass one and a plurality (i.e. one or more) of those steps, compositions of matter, groups of steps or group of compositions of matter.

Those skilled in the art will appreciate that the disclosure described herein is susceptible to variations and modifications other than those specifically described. It is to be understood that the disclosure includes all such variations and modifications. The disclosure also includes all of the steps, features, compositions and compounds referred to or indicated in this specification, individually or collectively, and any and all combinations or any two or more of said steps or features.

The present disclosure is not to be limited in scope by the specific embodiments described herein, which are intended for the purpose of exemplification only. Functionally-equivalent products, compositions and methods are clearly within the scope of the disclosure, as described herein.

Any example disclosed herein shall be taken to apply mutatis mutandis to any other example unless specifically stated otherwise.

Unless specifically defined otherwise, all technical and scientific terms used herein shall be taken to have the same meaning as commonly understood by one of ordinary skill in the art (e.g., in cell culture, molecular genetics, stem cell differentiation, immunology, immunohistochemistry, protein chemistry, and biochemistry).

Unless otherwise indicated, the surgical techniques utilized in the present disclosure are standard procedures, well known to those skilled in the art.

Methods of obtaining and enriching a population of mesenchymal lineage stem or precursor cells are known in the art. For example, enriched populations of mesenchymal lineage stem or precursor cells can be obtained by the use of flow cytometry and cell sorting procedures based on the use of cell surface markers that are expressed on mesenchymal lineage stem or precursor cells.

All documents cited or referenced herein, and all documents cited or referenced in herein cited documents, together with any manufacturer’s instructions, descriptions, product specifications, and product sheets for any products mentioned herein or in any document incorporated by reference herein, are hereby incorporated herein by reference in their entirety.

### Selected Definitions

The term “and/or”, e.g., “X and/or Y” shall be understood to mean either “X and Y” or “X or Y” and shall be taken to provide explicit support for both meanings or for either meaning.

As used herein, the term about, unless stated to the contrary, refers to +/- 10%, more preferably +/- 5%, of the designated value.

Throughout this specification the word “comprise”, or variations such as “comprises” or “comprising”, will be understood to imply the inclusion of a stated element, integer or step, or group of elements, integers or steps, but not the exclusion of any other element, integer or step, or group of elements, integers or steps.

As used herein, the singular form “a”, “an” and “the” include singular and plural references unless the context indicates otherwise.

By “isolated” or “purified” it is meant a cell which has been separated from at least some components of its natural environment. This term includes gross physical separation of the cells from its natural environment (e.g. removal from a donor). The term “isolated” includes alteration of the cell’s relationship with the neighboring cells with which it is in direct by, for example, dissociation. The term “isolated” does not refer to a cell which is in a tissue section. When used to refer to the population of cells, the term “isolated” includes populations of cells which result from proliferation of the isolated cells of the disclosure.

The terms “passage”, “passaging” or “sub-culture” are used in the context of the present disclosure to refer to known cell culture techniques that are used to keep cells alive and growing under cultured conditions for extended periods of time so that cell numbers can continually increase. The degree of sub-culturing a cell line has undergone is often expressed as “passage number,” which is generally used to refer to the number of times cells have been sub-cultured. In an example, one passage comprises removing non-adherent cells and leaving adherent mesenchymal lineage precursor or stem cells. Such mesenchymal lineage precursor or stem cells can then be dissociated from the substrate or flask (e.g., by using a protease such as trypsin or collagenase), media can be added, optional washing (e.g., by centrifugation) may be performed, and then the mesenchymal lineage precursor or stem cells can be re-plated or reseeded to one or more culture vessels containing a greater surface area in total. The mesenchymal lineage precursor or stem cells can then continue to expand in culture. In another example, methods of removing non-adherent cells include steps of non-enzymatic treatment (e.g., with EDTA). In an example, mesenchymal lineage precursor or stem cells are passaged at or near confluence (e.g., about 75% to about 95% confluence). In an example, the mesenchymal lineage precursor or stem cells are seeded at a concentration of about 10%, about 15%, or about 20% cells/ml of culture medium.

The term “medium” or “media” as used in the context of the present disclosure, includes the components of the environment surrounding cells in culture. It is envisaged that the media contributes to and/or provides the conditions suitable to allow cells to grow. Media may be solid, liquid, gaseous or a mixture of phases and materials. Media can include liquid growth media as well as liquid media that do not sustain cell growth. Exemplary gaseous media include the gaseous phase that cells growing on a petri dish or other solid or semisolid support are exposed to.

“Graft versus Host Disease (GvHD)” is an immunological disorder that is the major factor that limits the success and availability of allogeneic bone marrow or stem cell transplantation. GvHD occurs in acute (aGvHD) or chronic (cGvHD) forms. Acute GvHD usually manifests within 100 days following bone marrow or stem cell transplantation. Chronic GvHD generally manifests later than aGvHD (>100 days post transplantation) and has some features of autoimmune diseases. It may develop either de novo, following resolution of aGvHD or as an extension of aGvHD. Chronic GvHD can cause multiple, often debilitating symptoms, including widespread skin rashes, painful mouth ulcers, shortness of breath, and limb and joint pain. In an example, patients with cGvHD have impaired reconstitution of CD5+ B cells. In an example, cGvHD is refractory to steroid therapy. In an example, cGvHD is refractory to a biologic therapy. In an example cGvHD is refractory to steroid therapy and a biologic therapy.

As used herein, the terms “treating”, “treat” or “treatment” include administering a population of mesenchymal lineage stem or precursor cells and/or progeny thereof and/or soluble factors derived therefrom to thereby reduce or eliminate at least one symptom of cGvHD. In an example, treatment includes administering a population of culture expanded mesenchymal lineage precursor or stem cells. In an example, the treatment induces a partial response after treatment is initiated. In an example, the partial response is induced 28 days after treatment is initiated. In an example, the partial response is induced at least 28 after treatment is initiated. In an example, the partial response is induced at least 30 after treatment is initiated. In an example, the partial response is induced at least 2 months after treatment is initiated. In another example, the partial response is induced at least 3 months after treatment is initiated. In another example, the partial response is induced 28 to 56 days after treatment is initiated. In another example, the partial response is induced after two doses. In another example, the partial response is induced after two doses administered once weekly. In another example, the partial response is induced after two doses administered once weekly every two weeks. In another example, the partial response is induced after three doses or more.

In an example, a partial response is characterized by one or more or all of:
Reduction in Skin % BSA score of at least one point;
Reduction in mouth score of at least one point;
Reduction in eye score of at least one point;
Reduction in skin features score of at least one point;
Reduction in gastrointestinal tract score of at least one point;
Reduction in liver score of at least one point;
Reduction in lung symptom score of at least one point;
Reduction in lung FEV1 score of at least one point;
Reduction in joints and fascia score of at least one point;
Reduction in genital tract score of at least one point.

In an example, a partial response is characterized by a reduction in Skin % BSA score of at least one point. In another example, a partial response is characterized by a reduction in mouth score of at least one point. In another example, a partial response is characterized by a reduction in eye score of at least one point. In these examples, scores can be obtained using the NIH Consensus Criteria 2014 for GvHD (see for example, the Examples section below).

In another example, a partial response is characterized by one or more or all of:
Reduction in Skin % BSA score of at least one point;
Reduction in mouth score of at least one point;
Reduction in eye score of at least one point.

The term “prevent” or “preventing” as used herein include administering a population of mesenchymal lineage stem or precursor cells and/or progeny thereof and/or soluble factors derived therefrom to thereby stop or inhibit the development of at least one symptom of cGvHD.

There are various classification systems for characterizing GvHD (Lee, S., (2017) Blood., 129(1): 30-37). In an example, the NIH Consensus Criteria 2014 can be used for scoring outcomes disclosed herein (Jagasia et al., (2015) Biol Blood Marrow Transplant., 21:389-401). The components of the NIH Consensus Criteria 2014 are shown in the following table:

Organ Scoring of cGvHD

| | | | | |
|---|---|---|---|---|
| Organ | Score 0 | Score 1 | Score 2 | Score 3 |
| Skin % BSA¹ | No BSA involved | 1-18% BSA | 19-50% BSA | >50% BSA |
| Skin Features | No sclerotic features | N/A | Superficial sclerotic features, but not “hidebound” | Deep sclerotic features; “hidebound”; impaired mobility; ulceration |
| Mouth | No symptoms | Mild symptoms with disease signs but not limiting oral intake significantly | Moderate symptoms with disease signs with partial limitation of oral intake | Severe symptoms with disease signs with major limitation of oral intake |
| Eyes | No symptoms | Mild dry eye symptoms not affecting ADL (requirement of lubricant drops ≤3x/day) | Moderate dry eye symptoms partially affecting ADL (requiring lubricant drops >3x/day or punctal plugs) WITHOUT new vision impairment due to keratoconjunctivitis sicca (KCS) | Severe dry eye symptoms significantly affecting ADL (special eyewear to relieve pain) OR unable to work because of ocular symptoms OR loss of vision due to keratoconjunctivitis sicca (KCS) |
| GI Tract | No symptoms | Symptoms without significant weight loss (<5%) | Symptoms associated with mild to moderate weight loss (5-15%) within 3 months OR moderate diarrhea without significant interference with daily living | Symptoms associated with significant weight loss (>15%) within 3 months, requires nutritional supplement for most calorie needs OR esophageal dilation OR severe diarrhea with significant interference with daily living |
| Liver | Normal total bilirubin and ALT or AP <3 x ULN | Normal total bilirubin with ALT ≥3 to 5 x ULN or AP ≥3 x ULN | Elevated total bilirubin but ≤3 mg/dL or ALT >5 x ULN | Elevated total bilirubin but >3 mg/dL |
| Lungs Symptom Score | No symptoms | Mild symptoms (SOB after climbing one flight of steps) | Moderate symptoms (SOB after walking on flat ground) | Severe symptoms (SOB at rests; requires O2) |
| Lungs Lung Score | FEV1 ≥80% | FEV1 60-79% | FEV1 40-59% | FEV1 ≤39% |
| Joints and Fascia | No symptoms | Mild tightness of arms and legs, normal or mild decreased range of motion AND not affecting ADL | Tightness of arms or legs OR joint contractures, erythema thought to be due to fasciitis, moderate decrease of range of motion AND mild to moderate limitation of ADL | Contractures WITH significant decrease of range of motion AND significant limitation of ADL (unable to tie shoes, button shirts, dress shelf, etc.) |
| Genital Tract² | No signs | Mild signs and females with or without discomfort on exam | Moderate signs and may have signs of discomfort on exam | Severe signs with or without symptoms |
| Other features³ | No GVHD | Mild | Moderate | Severe |

In an example, a partial response is a decrease of ≥ 1 point on the organ-specific NIH Consensus Criteria 2014 score from the Table above. Accordingly, in an example, treatment induces ≥1 point decrease in Skin % BSA score. In another example, treatment induces ≥1 point decrease in mouth score. In another example, treatment induces ≥1 point decrease in eye score. In another example, treatment induces ≥1 point decrease in skin features score. In another example, treatment induces ≥1 point decrease in gastrointestinal tract score. In another example, treatment induces ≥1 point decrease in liver score. In another example, treatment induces ≥1 point decrease in lung symptom score. In another example, treatment induces ≥1 point decrease in lung FEV1 score. In another example, treatment induces ≥1 point decrease in joints and fascia score. In another example, treatment induces ≥1 point decrease in genital tract score.

In an example, the treatment induces a complete response after treatment is initiated. In an example, the complete response is induced 28 days after treatment is initiated. In an example, the complete response is induced at least 28 after treatment is initiated. In an example, the complete response is induced at least 30 after treatment is initiated. In an example, the complete response is induced at least 2 months after treatment is initiated. In another example, the complete response is induced at least 3 months after treatment is initiated. In another example, the complete response is induced 28 to 56 days after treatment is initiated. In another example, the complete response is induced after two doses. In another example, the complete response is induced after two doses administered once weekly. In another example, the complete response is induced after two doses administered once weekly every two weeks. In another example, the complete response is induced after three doses or more.

In an example, methods of the present disclosure inhibit cGvHD disease progression or disease complication in a subject. “Inhibition” of cGvHD disease progression or disease complication in a subject means preventing or reducing cGvHD progression and/or disease complication in the subject.

The term “subject” as used herein refers to a human subject. For example, the subject can be an adult. In another example, the subject can be a child. In another example, the subject can be an adolescent. Terms such as “subject”, “patient” or “individual” are terms that can, in context, be used interchangeably in the present disclosure.

Subjects treated according to the present disclosure may have symptoms indicative cGvHD. For example, a subject may have moderate or severe cGvHD. Exemplary symptoms include dry eye, >19% BSA, >50% BSA, maculopapular rash/erythema, papulosquamous lesions/ichthysis, hyperpigmentation, partial or major limitation of oral intake, diarrhea, elevated total bilirubin, reduced FEV1 and tightness or pain in joints.

In an example, the subject has previously failed at least one anti-TNF therapy. In an example, the subject has a contra-indication to biologic therapy. In another example, the subject has previously failed at least one steroid therapy.

In another example, the subject has had GvHD for >90 days. In another example, the subject has had GvHD for >100 days.

As used herein, the term “genetically unmodified” refers to cells that have not been modified by transfection with a nucleic acid. For the avoidance of doubt, in the context of the present disclosure a mesenchymal lineage precursor or stem cell transfected with a nucleic acid encoding Angl would be considered genetically modified.

### Mesenchymal Lineage Precursor Cells

As used herein, the term “mesenchymal lineage precursor or stem cell (MLPSC)” refers to undifferentiated multipotent cells that have the capacity to self-renew while maintaining multipotency and the capacity to differentiate into a number of cell types either of mesenchymal origin, for example, osteoblasts, chondrocytes, adipocytes, stromal cells, fibroblasts and tendons, or non-mesodermal origin, for example, hepatocytes, neural cells and epithelial cells. For the avoidance of doubt, a “mesenchymal lineage precursor cell” refers to a cell which can differentiate into a mesenchymal cell such as bone, cartilage, muscle and fat cells, and fibrous connective tissue.

The term “mesenchymal lineage precursor or stem cells” includes both parent cells and their undifferentiated progeny. The term also includes mesenchymal precursor cells, multipotent stromal cells, mesenchymal stem cells (MSCs), perivascular mesenchymal precursor cells, and their undifferentiated progeny.

Mesenchymal lineage precursor or stem cells can be autologous, allogeneic, xenogenic, syngenic or isogenic. Autologous cells are isolated from the same individual to which they will be reimplanted. Allogeneic cells are isolated from a donor of the same species. Xenogenic cells are isolated from a donor of another species. Syngenic or isogenic cells are isolated from genetically identical organisms, such as twins, clones, or highly inbred research animal models.

In an example, the mesenchymal lineage precursor or stem cells are allogeneic. In an example, the allogeneic mesenchymal lineage precursor or stem cells are culture expanded and cryopreserved.

Mesenchymal lineage precursor or stem cells reside primarily in the bone marrow, but have also shown to be present in diverse host tissues including, for example, cord blood and umbilical cord, adult peripheral blood, adipose tissue, trabecular bone and dental pulp. They are also found in skin, spleen, pancreas, brain, kidney, liver, heart, retina, brain, hair follicles, intestine, lung, lymph node, thymus, ligament, tendon, skeletal muscle, dermis, and periosteum; and are capable of differentiating into germ lines such as mesoderm and/or endoderm and/or ectoderm. Thus, mesenchymal lineage precursor or stem cells are capable of differentiating into a large number of cell types including, but not limited to, adipose, osseous, cartilaginous, elastic, muscular, and fibrous connective tissues. The specific lineage-commitment and differentiation pathway which these cells enter depends upon various influences from mechanical influences and/or endogenous bioactive factors, such as growth factors, cytokines, and/or local microenvironmental conditions established by host tissues.

The terms “enriched”, “enrichment” or variations thereof are used herein to describe a population of cells in which the proportion of one particular cell type or the proportion of a number of particular cell types is increased when compared with an untreated population of the cells (e.g., cells in their native environment). In one example, a population enriched for mesenchymal lineage precursor or stem cells comprises at least about 0.1% or 0.5% or 1% or 2% or 5% or 10% or 15% or 20% or 25% or 30% or 50% or 75% mesenchymal lineage precursor or stem cells. In this regard, the term “population of cells enriched for mesenchymal lineage precursor or stem cells” will be taken to provide explicit support for the term “population of cells comprising X% mesenchymal lineage precursor or stem cells”, wherein X% is a percentage as recited herein. The mesenchymal lineage precursor or stem cells can, in some examples, form clonogenic colonies, e.g. CFU-F (fibroblasts) or a subset thereof (e.g., 50% or 60% or 70% or 70% or 90% or 95%) can have this activity.

In an example of the present disclosure, the mesenchymal lineage precursor or stem cells are mesenchymal stem cells (MSCs). The MSCs may be a homogeneous composition or may be a mixed cell population enriched in MSCs. Homogeneous MSC compositions may be obtained by culturing adherent marrow or periosteal cells, and the MSCs may be identified by specific cell surface markers which are identified with unique monoclonal antibodies. A method for obtaining a cell population enriched in MSCs is described, for example, in U.S. Pat. No. 5,486,359. Alternative sources for MSCs include, but are not limited to, blood, skin, cord blood, muscle, fat, bone, and perichondrium. In an example, the MSCs are allogeneic. In an example, the MSCs are cryopreserved. In an example, the MSCs are culture expanded and cryopreserved.

In another example, the mesenchymal lineage precursor or stem cells are CD29+, CD54+, CD73+, CD90+, CD102+, CD105+, CD106+, CD166+, MHC1+ MSCs.

Isolated or enriched mesenchymal lineage precursor or stem cells can be expanded in vitro by culture. Isolated or enriched mesenchymal lineage precursor or stem cells can be cryopreserved, thawed and subsequently expanded in vitro by culture.

In one example, isolated or enriched mesenchymal lineage precursor or stem cells are seeded at 50,000 viable cells/cm² in culture medium (serum free or serum-supplemented), for example, alpha minimum essential media (αMEM) supplemented with 5% fetal bovine serum (FBS) and glutamine, and allowed to adhere to the culture vessel overnight at 37° C., 20% O₂. The culture medium is subsequently replaced and/or altered as required and the cells cultured for a further 68 to 72 hours at 37° C., 5% O₂.

As will be appreciated by those of skill in the art, cultured mesenchymal lineage precursor or stem cells are phenotypically different to cells in vivo. For example, in one embodiment they express one or more of the following markers, CD44, NG2, DC146 and CD140b. Cultured mesenchymal lineage precursor or stem cells are also biologically different to cells in vivo, having a higher rate of proliferation compared to the largely non-cycling (quiescent) cells in vivo.

In one example, the population of cells is enriched from a cell preparation comprising STRO-1+ cells in a selectable form. In this regard, the term “selectable form” will be understood to mean that the cells express a marker (e.g., a cell surface marker) permitting selection of the STRO-1+ cells. The marker can be STRO-1, but need not be. For example, as described and/or exemplified herein, cells (e.g., mesenchymal precursor cells) expressing STRO-2 and/or STRO-3 (TNAP) and/or STRO-4 and/or VCAM-1 and/or CD146 and/or 3G5 also express STRO-1 (and can be STRO-1bright). Accordingly, an indication that cells are STRO-1+ does not mean that the cells are selected solely by STRO-1 expression. In one example, the cells are selected based on at least STRO-3 expression, e.g., they are STRO-3+ (TNAP+).

Reference to selection of a cell or population thereof does not necessarily require selection from a specific tissue source. As described herein STRO-1+ cells can be selected from or isolated from or enriched from a large variety of sources. That said, in some examples, these terms provide support for selection from any tissue comprising STRO-1+ cells (e.g., mesenchymal precursor cells) or vascularized tissue or tissue comprising pericytes (e.g., STRO-1+ pericytes) or any one or more of the tissues recited herein.

In one example, the cells used in the present disclosure express one or more markers individually or collectively selected from the group consisting of TNAP+, VCAM-1+, THY-1+, STRO-2+, STRO-4+ (HSP-90β), CD45+, CD146+, 3G5+ or any combination thereof.

By “individually” is meant that the disclosure encompasses the recited markers or groups of markers separately, and that, notwithstanding that individual markers or groups of markers may not be separately listed herein the accompanying claims may define such marker or groups of markers separately and divisibly from each other.

By “collectively” is meant that the disclosure encompasses any number or combination of the recited markers or groups of markers, and that, notwithstanding that such numbers or combinations of markers or groups of markers may not be specifically listed herein the accompanying claims may define such combinations or sub- combinations separately and divisibly from any other combination of markers or groups of markers.

As used herein the term “TNAP” is intended to encompass all isoforms of tissue non-specific alkaline phosphatase. For example, the term encompasses the liver isoform (LAP), the bone isoform (BAP) and the kidney isoform (KAP). In one example, the TNAP is BAP. In one example, TNAP as used herein refers to a molecule which can bind the STRO-3 antibody produced by the hybridoma cell line deposited with ATCC on 19 Dec. 2005 under the provisions of the Budapest Treaty under deposit accession number PTA-7282.

Furthermore, in one example, the STRO-1+ cells are capable of giving rise to clonogenic CFU-F.

In one example, a significant proportion of the STRO-1+ cells are capable of differentiation into at least two different germ lines. Non-limiting examples of the lineages to which the STRO-1+ cells may be committed include bone precursor cells; hepatocyte progenitors, which are multipotent for bile duct epithelial cells and hepatocytes; neural restricted cells, which can generate glial cell precursors that progress to oligodendrocytes and astrocytes; neuronal precursors that progress to neurons; precursors for cardiac muscle and cardiomyocytes, glucose-responsive insulin secreting pancreatic beta cell lines. Other lineages include, but are not limited to, odontoblasts, dentin-producing cells and chondrocytes, and precursor cells of the following: retinal pigment epithelial cells, fibroblasts, skin cells such as keratinocytes, dendritic cells, hair follicle cells, renal duct epithelial cells, smooth and skeletal muscle cells, testicular progenitors, vascular endothelial cells, tendon, ligament, cartilage, adipocyte, fibroblast, marrow stroma, cardiac muscle, smooth muscle, skeletal muscle, pericyte, vascular, epithelial, glial, neuronal, astrocyte and oligodendrocyte cells.

In an example, mesenchymal lineage precursor or stem cells are obtained from a single donor, or multiple donors where the donor samples or mesenchymal lineage precursor or stem cells are subsequently pooled and then culture expanded.

Mesenchymal lineage precursor or stem cells encompassed by the present disclosure may also be cryopreserved prior to administration to a subject. In an example, mesenchymal lineage precursor or stem cells are culture expanded and cryopreserved prior to administration to a subject.

In an example, the present disclosure encompasses mesenchymal lineage precursor or stem cells as well as progeny thereof, soluble factors derived therefrom, and/or extracellular vesicles isolated therefrom. In another example, the present disclosure encompasses mesenchymal lineage precursor or stem cells as well as extracellular vesicles isolated therefrom. For example, it is possible to culture expand mesenchymal precursor lineage or stem cells of the disclosure for a period of time and under conditions suitable for secretion of extracellular vesicles into the cell culture medium. Secreted extracellular vesicles can subsequently be obtained from the culture medium for use in therapy.

The term “extracellular vesicles” as used herein, refers to lipid particles naturally released from cells and ranging in size from about 30 nm to as a large as 10 microns, although typically they are less than 200 nm in size. They can contain proteins, nucleic acids, lipids, metabolites, or organelles from the releasing cells (e.g., mesenchymal stem cells; STRO-1+ cells).

The term “exosomes” as used herein, refers to a type of extracellular vesicle generally ranging in size from about 30 nm to about 150 nm and originating in the endosomal compartment of mammalian cells from which they are trafficked to the cell membrane and released. They may contain nucleic acids (e.g., RNA; microRNAs), proteins, lipids, and metabolites and function in intercellular communication by being secreted from one cell and taken up by other cells to deliver their cargo.

### Culture Expansion of the Cells

In an example, mesenchymal lineage precursor or stem cells are culture expanded. “Culture expanded” mesenchymal lineage precursor or stem cells media are distinguished from freshly isolated cells in that they have been cultured in cell culture medium and passaged (i.e. sub-cultured). In an example, culture expanded mesenchymal lineage precursor or stem cells are culture expanded for about 4-10 passages. In an example, mesenchymal lineage precursor or stem cells are culture expanded for at least 5, at least 6, at least 7, at least 8, at least 9, at least 10 passages. For example, mesenchymal lineage precursor or stem cells can be culture expanded for at least 5 passages. In an example, mesenchymal lineage precursor or stem cells can be culture expanded for at least 5-10 passages. In an example, mesenchymal lineage precursor or stem cells can be culture expanded for at least 5-8 passages. In an example, mesenchymal lineage precursor or stem cells can be culture expanded for at least 5-7 passages. In an example, mesenchymal lineage precursor or stem cells can be culture expanded for more than 10 passages. In another example, mesenchymal lineage precursor or stem cells can be culture expanded for more than 7 passages. In these examples, stem cells may be culture expanded before being cryopreserved to provide an intermediate cryopreserved MLPSC population. In an example, compositions of the disclosure are prepared from an intermediate cryopreserved MLPSC population. For example, an intermediate cryopreserved MLPSC population can be further culture expanded prior to administration as is discussed further below. Accordingly, in an example, mesenchymal lineage precursor or stem cells are culture expanded and cryopreserved. In an embodiment of these examples, mesenchymal lineage precursor or stem cells can be obtained from a single donor, or multiple donors where the donor samples or mesenchymal lineage precursor or stem cells are subsequently pooled and then culture expanded. In an example, the culture expansion process comprises:
i. expanding by passage expansion the number of viable cells to provide a preparation of at least about 1 billion of the viable cells, wherein the passage expansion comprises establishing a primary culture of isolated mesenchymal lineage precursor or stem cells and then serially establishing a first non-primary (P1) culture of isolated mesenchymal lineage precursor or stem cells from the previous culture;
ii. expanding by passage expansion the P1 culture of isolated mesenchymal lineage precursor or stem cells to a second non-primary (P2) culture of mesenchymal lineage precursor or stem cells; and,
iii. preparing and cryopreserving an in-process intermediate mesenchymal lineage precursor or stem cells preparation obtained from the P2 culture of mesenchymal lineage precursor or stem cells; and,
iv. thawing the cryopreserved in-process intermediate mesenchymal lineage precursor or stem cells preparation and expanding by passage expansion the in-process intermediate mesenchymal lineage precursor or stem cells preparation.

In an example, the expanded mesenchymal lineage precursor or stem cell preparation has an antigen profile and an activity profile comprising:
i. less than about 0.75% CD45+ cells;
ii. at least about 95% CD105+ cells;
iii. at least about 95% CD166+ cells.

In an example, the expanded mesenchymal lineage precursor or stem cell preparation is capable of inhibiting IL2Ra expression by CD3/CD28-activated PBMCs by at least about 30% relative to a control.

In an example, culture expanded mesenchymal lineage precursor or stem cells are culture expanded for about 4-10 passages, wherein the mesenchymal lineage precursor or stem cells have been cryopreserved after at least 2 or 3 passages before being further culture expanded. In an example, mesenchymal lineage precursor or stem cells are culture expanded for at least 1, at least 2, at least 3, at least 4, at least 5 passages, cryopreserved and then further culture expanded for at least 1, at least 2, at least 3, at least 4, at least 5 passages before being administered or further cryopreserved.

In an example, the majority of mesenchymal lineage precursor or stem cells in compositions of the disclosure are of about the same generation number (i.e., they are within about 1 or about 2 or about 3 or about 4 cell doublings of each other). In an example, the average number of cell doublings in the present compositions is about 20 to about 25 doublings. In an example, the average number of cell doublings in the present compositions is about 9 to about 13 (e.g., about 11 or about 11.2) doublings arising from the primary culture, plus about 1, about 2, about 3, or about 4 doublings per passage (for example, about 2.5 doublings per passage). Exemplary average cell doublings in present compositions are any of about 13.5, about 16, about 18.5, about 21, about 23.5, about 26, about 28.5, about 31, about 33.5, and about 36 when produced by about 1, about 2, about 3, about 4, about 5, about 6, about 7, about 8, about 9, and about 10 passages, respectively.

The process of mesenchymal lineage precursor or stem cell isolation and ex vivo expansion can be performed using any equipment and cell handing methods known in the art. Various culture expansion embodiments of the present disclosure employ steps that require manipulation of cells, for example, steps of seeding, feeding, dissociating an adherent culture, or washing. Any step of manipulating cells has the potential to insult the cells. Although mesenchymal lineage precursor or stem cells can generally withstand a certain amount of insult during preparation, cells are preferably manipulated by handling procedures and/or equipment that adequately performs the given step(s) while minimizing insult to the cells.

In an example, mesenchymal lineage precursor or stem cells are washed in an apparatus that includes a cell source bag, a wash solution bag, a recirculation wash bag, a spinning membrane filter having inlet and outlet ports, a filtrate bag, a mixing zone, an end product bag for the washed cells, and appropriate tubing, for example, as described in US 6,251,295, which is hereby incorporated by reference.

In an example, a mesenchymal lineage precursor or stem cell composition according to the present disclosure is 95% homogeneous with respect to being CD105 positive and CD166 positive and being CD45 negative. In an example, this homogeneity persists through ex vivo expansion; i.e. though multiple population doublings. In an example, the composition comprises at least one therapeutic dose of mesenchymal lineage precursor or stem cells and the mesenchymal lineage precursor or stem cells comprise less than about 1.25% CD45+ cells, at least about 95% CD105+ cells, and at least about 95% CD166+ cells. In an example, this homogeneity persists after cryogenic storage and thawing, where the cells also generally have a viability of about 70% or more.

In an example, compositions of the disclosure comprise mesenchymal lineage precursor or stem cells which express substantial levels of TNFR1, for example greater than 13 pg of TNFR1 per million mesenchymal lineage precursor or stem cells. In an example, this phenotype is stable throughout ex vivo expansion and cryogenic storage. In an example, expression of levels of TNFR1 in the range of about 13 to about 179 pg (e.g. about 13 pg to about 44 pg) per million mesenchymal lineage precursor or stem cells is associated with a desirous therapeutic potential which also persists through ex vivo expansion and cryopreservation.

In an example, the culture expanded mesenchymal lineage precursor or stem cells express Tumor necrosis factor receptor 1 (TNFR1) in an amount of at least 110 pg/ml. For example, the mesenchymal lineage precursor or stem cells can express TNFR1 in an amount of at least 150 pg/ml, or at least 200 pg/ ml, or at least 250 pg/ml, or at least 300 pg/ml, or at least 320 pg/ml, or at least 330 pg/ml, or at least 340 pg/ml, or at least 350 pg/ml.

In an example, the mesenchymal lineage precursor or stem cells express TNFR1 in an amount of at least 13 pg/10⁶ cells. For example, the mesenchymal lineage precursor or stem cells express TNFR1 in an amount of at least 15 pg/10⁶ cells, or at least 20 pg/10⁶ cells, or at least 25 pg/10⁶ cells, or at least 30 pg/10⁶ cells, or at least 35 pg/10⁶ cells, or at least 40 pg/10⁶ cells, or at least 45 pg/10⁶ cells, or at least 50 pg/10⁶ cells.

In another example, mesenchymal lineage precursor or stem cells disclosed herein inhibit IL-2Rα expression on T-cells. In an example, mesenchymal lineage precursor or stem cells can inhibit IL-2Rα expression by at least about 30%, alternatively at least about 35%, alternatively at least about 40%, alternatively at least about 45%, alternatively at least about 50%, alternatively at least about 55%, alternatively at least about 60.

In an example, compositions of the disclosure comprise at least one therapeutic dose of mesenchymal lineage precursor or stem cells which, for example, can comprise at least about 100 million cells or about 125 million cells.

### Modification of the Cells

The mesenchymal lineage precursor or stem cells of the present disclosure may be altered in such a way that upon administration, lysis of the cell is inhibited. Alteration of an antigen can induce immunological non-responsiveness or tolerance, thereby preventing the induction of the effector phases of an immune response (e.g., cytotoxic T cell generation, antibody production etc.) which are ultimately responsible for rejection of foreign cells in a normal immune response. Antigens that can be altered to achieve this goal include, for example, MHC class I antigens, MHC class II antigens, LFA-3 and ICAM-1.

The mesenchymal lineage precursor or stem cells may also be genetically modified to express proteins of importance for the differentiation and/or maintenance of striated skeletal muscle cells. Exemplary proteins include growth factors (TGF-β, insulin-like growth factor 1 (IGF-1), FGF), myogenic factors (e.g. myoD, myogenin, myogenic factor 5 (Myf5), myogenic regulatory factor (MRF)), transcription factors (e.g. GATA-4), cytokines (e.g. cardiotropin-1), members of the neuregulin family (e.g. neuregulin 1, 2 and 3) and homeobox genes (e.g. Csx, tinman and NKx family).

### Compositions of the Disclosure

In one example of the present disclosure the mesenchymal lineage precursor or stem cells and/or progeny thereof and/or soluble factor derived therefrom are administered in the form of a composition. In one example, such a composition comprises a pharmaceutically acceptable carrier and/or excipient. Accordingly, in an example, compositions of the disclosure can comprise culture expanded mesenchymal lineage precursor or stem cells.

The terms “carrier” and “excipient” refer to compositions of matter that are conventionally used in the art to facilitate the storage, administration, and/or the biological activity of an active compound (see, e.g., Remington’s Pharmaceutical Sciences, 16th Ed., Mac Publishing Company (1980). A carrier may also reduce any undesirable side effects of the active compound. A suitable carrier is, for example, stable, e.g., incapable of reacting with other ingredients in the carrier. In one example, the carrier does not produce significant local or systemic adverse effect in recipients at the dosages and concentrations employed for treatment.

Suitable carriers for the present disclosure include those conventionally used, e.g., water, saline, aqueous dextrose, lactose, Ringer’s solution, a buffered solution, hyaluronan and glycols are exemplary liquid carriers, particularly (when isotonic) for solutions. Suitable pharmaceutical carriers and excipients include starch, cellulose, glucose, lactose, sucrose, gelatin, malt, rice, flour, chalk, silica gel, magnesium stearate, sodium stearate, glycerol monostearate, sodium chloride, glycerol, propylene glycol, water, ethanol, and the like.

In another example, a carrier is a media composition, e.g., in which a cell is grown or suspended. For example, such a media composition does not induce any adverse effects in a subject to whom it is administered.

Exemplary carriers and excipients do not adversely affect the viability of a cell and/or the ability of a cell to reduce, prevent or delay metabolic syndrome and/or obesity.

In one example, the carrier or excipient provides a buffering activity to maintain the cells and/or soluble factors at a suitable pH to thereby exert a biological activity, e.g., the carrier or excipient is phosphate buffered saline (PBS). PBS represents an attractive carrier or excipient because it interacts with cells and factors minimally and permits rapid release of the cells and factors, in such a case, the composition of the disclosure may be produced as a liquid for direct application to the blood stream or into a tissue or a region surrounding or adjacent to a tissue, e.g., by injection.

The mesenchymal lineage precursor or stem cells and/or progeny thereof and/or soluble factor derived therefrom can also be incorporated or embedded within scaffolds that are recipient-compatible and which degrade into products that are not harmful to the recipient. These scaffolds provide support and protection for cells that are to be transplanted into the recipient subjects. Natural and/or synthetic biodegradable scaffolds are examples of such scaffolds.

A variety of different scaffolds may be used successfully in the practice of the disclosure. Exemplary scaffolds include, but are not limited to biological, degradable scaffolds. Natural biodegradable scaffolds include collagen, fibronectin, and laminin scaffolds. Suitable synthetic material for a cell transplantation scaffold should be able to support extensive cell growth and cell function. Such scaffolds may also be resorbable. Suitable scaffolds include polyglycolic acid scaffolds, (e.g., as described by Vacanti, et al. J. Ped. Surg. 23:3-9 1988; Cima, et al. Biotechnol. Bioeng. 38:145 1991; Vacanti, et al. Plast. Reconstr. Surg. 88:753-9 1991); or synthetic polymers such as polyanhydrides, polyorthoesters, and polylactic acid.

In another example, the mesenchymal lineage precursor or stem cells and/or progeny thereof and/or soluble factor derived therefrom may be administered in a gel scaffold (such as Gelfoam from Upjohn Company).

The compositions described herein may be administered alone or as admixtures with other cells. The cells of different types may be admixed with a composition of the disclosure immediately or shortly prior to administration, or they may be co-cultured together for a period of time prior to administration.

In one example, the composition comprises an effective amount or a therapeutically or prophylactically effective amount of mesenchymal lineage precursor or stem cells and/or progeny thereof and/or soluble factor derived therefrom. For example, the composition comprises about 1x10⁵ mesenchymal lineage precursor or stem cells to about 1x10⁹ mesenchymal lineage precursor or stem cells or about 1.25x10³ mesenchymal lineage precursor or stem cells to about 1.25x10⁷ mesenchymal lineage precursor or stem cells/kg (80 kg subject). In another example, the composition comprises about 1x10⁶ mesenchymal lineage precursor or stem cells to about 3x10⁶ mesenchymal lineage precursor or stem cells/kg (80 kg subject).

The exact amount of cells to be administered is dependent upon a variety of factors, including the age, weight, and sex of the subject, and the extent and severity of the disorder being treated.

In an example, 50 x 10⁶ to 200 x 10⁷ mesenchymal lineage precursor or stem cells are administered. In other examples, 60 x 10⁶ to 200 x 10⁶ cells or 75 x 10⁶ to 150 x 10⁶ mesenchymal lineage precursor or stem cells are administered. In an example, 75 x 10⁶ mesenchymal lineage precursor or stem cells are administered. In another example, 150 x 10⁶ mesenchymal lineage precursor or stem cells are administered.

In an example, , the composition comprises greater than 5.00x10⁶ viable cells/mL. In another example, the composition comprises greater than 5.50x10⁶ viable cells/mL. In another example, the composition comprises greater than 6.00x10⁶ viable cells/mL. In another example, the composition comprises greater than 6.50x10⁶ viable cells/mL. In another example, the composition comprises greater than 6.68x10⁶ viable cells/mL.

In an example, the mesenchymal lineage precursor or stem cells comprise at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 99% of the cell population of the composition.

Compositions of the disclosure may be cryopreserved. Cryopreservation of mesenchymal lineage precursor or stem cells can be carried out using slow-rate cooling methods or ‘fast’ freezing protocols known in the art. Preferably, the method of cryopreservation maintains similar phenotypes, cell surface markers and growth rates of cryopreserved cells in comparison with unfrozen cells.

The cryopreserved composition may comprise a cryopreservation solution. The pH of the cryopreservation solution is typically 6.5 to 8, preferably 7.4.

The cryopreservation solution may comprise a sterile, non-pyrogenic isotonic solution such as, for example, PlasmaLyte A™. 100 mL of PlasmaLyte A™ contains 526 mg of sodium chloride, USP (NaCl); 502 mg of sodium gluconate (C₆H₁₁NaO₇); 368 mg of sodium acetate trihydrate, USP (C₂H₃NaO₂•3H₂O); 37 mg of potassium chloride, USP (KCl); and 30 mg of magnesium chloride, USP (MgCl₂•6H₂O). It contains no antimicrobial agents. The pH is adjusted with sodium hydroxide. The pH is 7.4 (6.5 to 8.0).

The cryopreservation solution may comprise Profreeze™. The cryopreservation solution may additionally or alternatively comprise culture medium, for example, αMEM.

To facilitate freezing, a cryoprotectant such as, for example, dimethylsulfoxide (DMSO), is usually added to the cryopreservation solution. Ideally, the cryoprotectant should be nontoxic for cells and patients, nonantigenic, chemically inert, provide high survival rate after thawing and allow transplantation without washing. However, the most commonly used cryoprotector, DMSO, shows some cytotoxicity . Hydroxylethyl starch (HES) may be used as a substitute or in combination with DMSO to reduce cytotoxicity of the cryopreservation solution.

The cryopreservation solution may comprise one or more of DMSO, hydroxyethyl starch, human serum components and other protein bulking agents. In one example, the cryopreserved solution comprises about 5% human serum albumin (HSA) and about 10% DMSO. The cryopreservation solution may further comprise one or more of methycellulose, polyvinyl pyrrolidone (PVP) and trehalose.

In one embodiment, cells are suspended in 42.5% Profreeze™/50% αMEM/7.5% DMSO and cooled in a controlled-rate freezer.

The cryopreserved composition may be thawed and administered directly to the subject or added to another solution, for example, comprising HA. Alternatively, the cryopreserved composition may be thawed and the mesenchymal lineage precursor or stem cells resuspended in an alternate carrier prior to administration.

In an example, cellular compositions of the disclosure can comprise Plasma-Lyte A, dimethyl sulfoxide (DMSO) and human serum albumin (HSA). For example, compositions of the disclosure may comprise Plasma-Lyte A (70%), DMSO (10%), HSA (25%) solution, the HSA solution comprising 5% HSA and 15% buffer.

In an example, the compositions described herein may be administered as a single dose.

In some examples, the compositions described herein may be administered over multiple doses. For example, at least 2, at least 3, at least 4, at least 5, at least 6, at least 7, at least 8, at least 9, at least 10 doses.

In some examples, the cells are contained within a chamber that does not permit the cells to exit into a subject’s circulation but permits factors secreted by the cells to enter the circulation. In this manner soluble factors may be administered to a subject by permitting the cells to secrete the factors into the subject’s circulation. Such a chamber may equally be implanted at a site in a subject to increase local levels of the soluble factors.

In an example, mesenchymal lineage precursor or stem cells may be administered intravenously. In another example, mesenchymal lineage precursor or stem cells are administered once weekly. For example, mesenchymal lineage precursor or stem cells can be administered once weekly every two weeks. In an example, mesenchymal lineage precursor or stem cells can be administered once monthly. In an example, two doses of mesenchymal lineage precursor or stem cells are administered once weekly. In another example, two doses of mesenchymal lineage precursor or stem cells are administered once weekly every two weeks. For example, two doses of mesenchymal lineage precursor or stem cells can be administered once weekly every two weeks before subsequent doses are administered once monthly. In an embodiment of this example, doses are administered monthly for a further one, two, three, four, five, six, seven or more months.

It will be appreciated by persons skilled in the art that numerous variations and/or modifications may be made to the above-described embodiments, without departing from the broad general scope of the present disclosure. The present embodiments are, therefore, to be considered in all respects as illustrative and not restrictive.

The following specific examples are to be construed as merely illustrative, and not limitative of the remainder of the disclosure in any way whatsoever. Without further elaboration, it is believed that one skilled in the art can, based on the description herein, utilize the present invention to its fullest extent.

### EXAMPLES

### Ex Vivo Culture Expanded Adult Allogeneic Bone Marrow Derived Mesenchymal Stem Cells (MSCs), for the Treatment of Chronic Graft Versus Host Disease (cGvHD)

### Composition

The composition is comprised of culture expanded mesenchymal stromal cells (ceMSC) isolated from the bone marrow of healthy adult donors. The final composition comprises ceMSC formulated in Plasma-Lyte A (70%), dimethyl sulfoxide (DMSO, 10%) and human serum albumin (HSA) (25%) solution (20%, comprising 5% HSA and 15% buffer) at a concentration of ≥6.68x106 viable cells/mL. Each dose vial contains 3.8 mL of cryopreserved cell suspension (total cells per vial ≥25x10⁶).

### Objectives

### Primary Objective

To evaluate the efficacy of repeated doses of the above composition administered intravenously over 6 months from its first date of administration in subjects with cGvHD that have failed to respond to prior treatment with conventional therapies.

### Secondary Objectives

To evaluate the effect of the above composition on survival.

### Scoring and Efficacy Assessments

Chronic GvHD organ scoring.

Organ Scoring of Chronic GVHD

| | | | | |
|---|---|---|---|---|
| Organ | Score 0 | Score 1 | Score 2 | Score 3 |
| Skin % BSA¹ | No BSA involved | 1-18% BSA | 19-50% BSA | >50% BSA |
| Skin Features | No sclerotic features | N/A | Superficial sclerotic features, but not “hidebound” | Deep sclerotic features; “hidebound;” impaired mobility; ulceration |
| Mouth | No symptoms | Mild symptoms with disease signs but not limiting oral intake significantly | Moderate symptoms with disease signs with partial limitation of oral intake | Severe symptoms with disease signs with major limitation of oral intake |
| Eyes | No symptoms | Mild dry eye symptoms not affecting ADL (requirement of lubricant drops ≤ 3x/day) | Moderate dry eye symptoms partially affecting ADL (requiring lubricant drops > 3x/day or punctal plugs) WITHOUT new vision impairment due to keratoconjunctivitis sicca (KCS) | Severe dry eye symptoms significantly affecting ADL (special eyewear to relieve pain) OR unable to work because of ocular symptoms OR loss of vision due to keratoconjunctivitis sicca (KCS) |
| GI Tract | No symptoms | Symptoms without significant weight loss (< 5%) | Symptoms associated with mild to moderate weight loss (5-15%) within 3 months OR moderate diarrhea without significant interference with daily living | Symptoms associated with significant weight loss (> 15%) within 3 months, requires nutritional supplement for most calorie needs OR esophageal dilation OR severe diarrhea with significant interference with daily living. |
| Liver | Normal total bilirubin and ALT or AP < 3 x ULN | Normal total bilirubin with ALT ≥ 3 to 5 x ULN or AP ≥ 3 x ULN | Elevated total bilirubin but ≤ 3 mg / dL or ALT > 5 x ULN | Elevated total bilirubin > 3 mg / dL |
| Lungs Symptom Score: | No symptoms | Mild symptoms (SOB after climbing one flight of steps) | Moderate symptoms (SOB after walking on flat ground) | Severe symptoms (SOB at rests; requires O2) |
| Lungs Lung Score: | FEV1 ≥ 80% | FEV1 60-79% | FEV1 40-59% | FEV1 ≤ 39% |
| Joints and Fascia | No symptoms | Mild tightness of arms or legs, normal or mild decreased range of motion AND not affecting ADL | Tightness of arms or legs OR joint contractures, erythema thought to be due to fasciitis, moderate decrease of range of motion AND mild to moderate limitation of ADL | Contractures WITH significant decrease of range of motion AND significant limitation of ADL (unable to tie shoes, button shirts, dress self, etc.) |
| Genital Tract² | No signs | Mild signs and females with or without discomfort on exam | Moderate signs and may have signs of discomfort on exam | Severe signs with or without symptoms |
| Other Features³ | No GVHD | Mild | Moderate | Severe |

Chronic GvHD response criteria.

| | |
|---|---|
| Abbreviation | Definition |
| CR | Complete Response: full resolution of cGVHD. |
| PR | Partial Response: decrease of ≥ 1 point on the organ-specific NIH cGVHD score. |
| SD | Stable Disease: no change in cGVHD |
| PD | Progressive Disease: progressing cGVHD during or up to 8 weeks post MSC treatment. |

Treatment success defined as the subject alive and assessed graded as a CR or PR at 6 months post treatment initiation.

### Subjects

Subject will be treated with intravenous (IV) stem cell composition at a dose of 2 x 10⁶ MSC/kg (actual body weight at screening) once weekly(± 1 day) for 2 weeks, for the first month. If eligible, continued therapy of 2 x10⁶ MSC/kg will be provided once monthly for up to 5 additional months.

### Assessments

GvHD assessments will be performed at screening and then monthly (±2 days for Months 2-6 then ±7 days for Months 7- 12) thereafter until 12 months post the initial stem cells infusion. Determination of whether subjects may go on to receive Continued Cell Therapy will be determined by the Investigator based on Subject’s cGvHD assessment/grading performed at 4 weeks (Day 28 (±2 days)) post 1 dose of composition (Day 0).

### Month One (Day 28 (±2 Days)) Therapy Assessment

A therapy assessment will be performed at four weeks (Day 28 (±2 days}) from the first infusion of cell composition to determine treatment response and to determine whether Subject will go on to receive Continued Therapy. The Day 28 therapy assessment must be at least 24 hours after the last dose of cell composition. Continued Therapy will be allowed for Subject according to response to treatment, as described below.

### Months 2, 3, 4, 5, 6, 7, 8, 9, 10, 11 and 12 Therapy Assessments

A therapy assessment will be performed once monthly (±2 days for Months 2 -6 then ±7 days for Months 7 - 12) to determine treatment response. Each monthly therapy assessment must be at least 24 hours after the last dose of cell composition.

### Continued Therapy

Subject may receive additional once-monthly infusions of cell composition at the same initial dose of 2 x10⁶ MSC/kg actual body weight at screening, which would begin within one week after the Initial Therapy assessment performed at 4 weeks (Day 28 (±2 days)) post initial treatment. Continued Therapy infusions will be given once monthly (±2 days) for up to 5 additional months.

### Analysis

Patient#1 AO 030-02C:
Patient completed 4/7 cell composition infusions.
   Baseline Assessment:
      Organ involvement: Skin, mouth, eyes
      Skin features: maculopapular rash/erythema, papulosquamous lesions/ichthysis
      Other skin findings: hyperpigmentation, hair involvement
      Skin % BSA score = 3 (greater than 50%BSA)
      Eye score = 1 (Mild dry eye symptoms not affecting ADL(requirement of lubricant drops less than or equal to 3x/day)
      Mouth score = 1 (Mild symptoms with disease signs but not limiting oral intake significantly)
      Maximum Grade of cGvHD (according to best clinical judgement) = Severe
   Day 28 response assessed as a Partial Response:
      Organ involvement: Skin, eyes (no mouth involvement)
      Skin features: maculopapular rash/erythema(baseline finding) papulosquamous lesions or ichthyosis (day 28 finding)
      Other skin findings: hair involvement (improved), nail involvement (improved)
      Skin % BSA score = 1 (1-18% BSA)
      Eye score = 1 (Mild dry eye symptoms not affecting ADL(requirement of lubricant drops less than or equal to 3x/day
      Subjective Clinician Opinion of Severity: Mild

Patient#2 CL 054-001:
Completed 5/7 cell composition infusions.
   Baseline Assessment:
      Organ involvement: Skin, mouth, eyes
      Skin features: maculopapular rash/erythema
      Other skin findings: severe/generalized pruritis, hair involvement, nail involvement
      Skin % BSA score = 1 (1-18% BSA)
      Eye score = 1 (Mild dry eye symptoms not affecting ADL(requirement of lubricant drops less than or equal to 3x/day)
      Mouth score = 1 (Mild symptoms with disease signs but not limiting oral intake significantly)
      Maximum Grade of cGvHD (according to best clinical judgement) = Moderate
   Day 28 response assessed as a Partial Response:
      Organ involvement: Skin, eyes (no mouth involvement)
      Skin features: papulosquamous lesions or ichthyosis
      Other skin findings: hair involvement (improved), nail involvement
      Skin % BSA score = 1 (1-18% BSA)
      Eye score = 1 (Mild dry eye symptoms not affecting ADL(requirement of lubricant drops less than or equal to 3x/day)
      Subjective Clinician Opinion of Severity: Mild
   Month 2: assessed as a Partial Response:
      Organ involvement: Skin, eyes (no mouth involvement)
      Skin features: few red areas over fingers, upper and lower back chronic areas resolved w/ 2-3 small round patches, ears w/some mild redness, no rash over scalp and hair thicker
      Other skin findings: hair involvement, nail involvement
      Skin % BSA score = 1 (1-18% BSA)
      Eye score = 1 (Mild dry eye symptoms not affecting ADL(requirement of lubricant drops less than or equal to 3x/day)
      Subjective Clinician Opinion of Severity: Mild

Patient#3 PF 055-001:
Completed 2/7 cell composition infusions.
   Baseline Assessment:
      Skin % BSA score = 2 (19-50% BSA)
      Eye score = 2 (Moderate dry eye symptoms partially affecting ADL (requiring lubricant drops greater than 3x/day or punctal plugs) without new vision impairment due to keratoconjunctivitis sicca)
      Mouth score = 1 (Mild symptoms with disease signs but not limiting oral intake significantly)
      Maximum Grade of cGvHD (according to best clinical judgement) = Moderate
   Day 28 response assessed as a Partial Response:
      The patient’s skin (main organ involved) improved dramatically. Numerous lesions at baseline were reduced to 2.

It will be appreciated by persons skilled in the art that numerous variations and/or modifications may be made to the disclosure as shown in the specific embodiments without departing from the spirit or scope of the disclosure as broadly described. The present embodiments are, therefore, to be considered in all respects as illustrative and not restrictive.

All publications discussed above are incorporated herein in their entirety.

Any discussion of documents, acts, materials, devices, articles or the like which has been included in the present specification is solely for the purpose of providing a context for the present disclosure. It is not to be taken as an admission that any or all of these matters form part of the prior art base or were common general knowledge in the field relevant to the present disclosure as it existed before the priority date of each claim of this application.

The present application claims priority from Australian Provisional Patent Application 2020900472 filed 19 Feb. 2020, the entire contents of which are incorporated herein by reference.

1. A method of treating or preventing chronic Graft versus Host Disease (cGvHD) in a human subject in need thereof, the method comprising administering to the subject a composition comprising culture expanded mesenchymal lineage precursor or stem cells (MLPSCs).

2. The method of claim 1, wherein the MLPSCs have been cryopreserved and thawed.

3. The method of claim 1 or claim 2, wherein the MLPSCs are culture expanded from an intermediate cryopreserved MLPSCs population.

4. The method according to any one of claims 1 to 3, wherein the MLPSCs are culture expanded for at least about 5 passages.

5. The method of any one of claims 1 to 4, wherein the MLPSCs express at least 13 pg TNFR1 per million MLPSCs.

6. The method of any one of claims 1 to 4, wherein the MLPSCs express about 13 pg to about 44 pg TNFR1 per million MLPSCs.

7. The method of any one of claims 1 to 6, wherein said culture expansion comprises at least 20 population doublings.

8. The method of any one of claims 1 to 6, wherein said culture expansion comprises at least 30 population doublings.

9. The method according to any one of claims 1 to 8, wherein the subject is refractory to steroid immunosuppressant and/or a biologic therapy.

10. The method according to any one of claims 1 to 9, wherein the subject has at least a partial response after 28 days of treatment.

11. The method according to any one of claims 1 to 9, wherein the subject has at least a partial response at least 28 to 90 days after treatment.

12. The method of claim 10 or 11wherein a partial response is characterized by one or more or all of:
Reduction in Skin % BSA score of at least one point;
Reduction in mouth score of at least one point;
Reduction in eye score of at least one point;
Reduction in skin features score of at least one point;
Reduction in gastrointestinal tract score of at least one point;
Reduction in liver score of at least one point;
Reduction in lung symptom score of at least one point;
Reduction in lung FEV1 score of at least one point;
Reduction in joints and fascia score of at least one point;
Reduction in genital tract score of at least one point.

13. The method of claim 10 or 11 wherein a partial response is characterized by one or more or all of:
Reduction in Skin % BSA score of at least one point;
Reduction in mouth score of at least one point;
Reduction in eye score of at least one point.

14. The method according to any one of claims 1 to 13, wherein the MLPSCs are administered intravenously.

15. The method according to any one of claims 1 to 14, wherein the MLPSCs are mesenchymal stem cells (MSCs).

16. The method according to any one of claims 1 to 15, wherein the MLPSCs are allogeneic.

17. The method according to any one of claims 1 to 16 which comprises administering between 10 × 10⁶ and 2 × 10⁸ cells per dose.

18. The method according to any one of claims 1 to 16 which comprises administering between 20 × 10⁶ and 1 × 10⁷ cells per dose.

19. The method according to claim 17 or 18, wherein the subject receives at least two doses.

20. The method according to claim 17 or 18, wherein the subject receives at least 2, 3, 4, 5, 6, 7, 8, 9 or 10 doses.

21. The method of claim 19 or 20, wherein the first two doses are administered weekly for two weeks.

22. The method of claim 19 or 20, wherein the first two doses are administered weekly every two weeks.

23. The method of claim 22, wherein third and subsequent doses are administered monthly.

24. The method according to any one of claims 1 to 23, wherein the composition further comprises Plasma-Lyte A, dimethyl sulfoxide (DMSO), human serum albumin (HSA).

25. The method according to any one of claims 1 to 23, wherein the composition further comprises Plasma-Lyte A (70%), DMSO (10%), HSA (25%) solution, the HSA solution comprising 5% HSA and 15% buffer.

26. The method according to any one of claims 1 to 25, wherein the composition comprises greater than 6.68×10⁶ viable cells/mL.
